(19) United States
(12) Patent Application Publication
(10) Pub. No.: US 2015/0041631 A1
(43) Pub. Date: Feb. 12, 2015

(54) READOUT & SIGNAL TRANSDUCTION (ROST) COMPONENT FOR POC DEVICES

(71) Applicant: WILLIAM MARSH RICE UNIVERSITY, Houston, TX (US)

(72) Inventors: Tomasz Stanislaw Tkaczyk, Houston, TX (US); Rebecca Richards-Kortum, Houston, TX (US)

(21) Appl. No.: 14/305,790

(22) Filed: Jun. 16, 2014

Related U.S. Application Data
(63) Continuation of application No. PCT/US12/70185, filed on Dec. 17, 2012.
(60) Provisional application No. 61/576,851, filed on Dec. 16, 2011.

(51) Int. Cl. G01N 33/483; G02B 7/02; G01N 21/27; G01N 21/64; G01N 21/47; G02B 7/00; F21V 33/00

(52) U.S. Cl. CPC G01N 33/483; G02B 7/003; G02B 7/026; F21V 33/0068; G01N 21/6486; G01N 21/47; G01N 21/27
USPC 250/216; 359/811; 359/819; 362/382

(57) ABSTRACT

A readout and signal transduction (ROST) component includes a universal fixture adapted to receive and align one or more optical elements and one or more opto-mechanical elements, wherein the optical elements and one or more opto-mechanical elements are stacked in the universal fixture, one or more optical elements configured for integration into the universal fixture; and one or more opto-mechanical elements configured for integration into the universal fixture.

### CROSS-REFERENCE TO RELATED APPLICATION

The present application is a continuation of PCT/US12/70185 filed Dec. 17, 2012 which claims priority to U.S. Provisional Patent Application Ser. No. 61/576,851 filed Dec. 16, 2011, which is incorporated herein by reference.

### BACKGROUND

Optical methods for readout and signal transduction have many advantages in point-of-care (“POC”) diagnostic tests, including compatibility with benchtop assays, environmental stability, and potential for multiplexed detection of several targets. However, POC test developers currently lack access to high quality, low-cost optics, compatible with fluorescence, scattering, reflectance, or transmission assays. As a result, most lateral flow assays provide only a binary “yes or no” diagnosis, and microfluidic cartridges still require a traditional microscope or bulky reader for readout. In order to overcome this barrier and advance the utility and performance of current and emerging POC diagnostics, a high quality, cost-effective optical readout and signal transduction component is required.

Anemia provides an example of the need for low-cost point-of-care diagnostic tests. Hemoglobin concentration assessment is the most commonly performed laboratory test worldwide, driven by the impact of anemia, and is necessary for all healthcare systems. Anemia affects an estimated quarter of the world's population and can be caused by iron deficiency, malnutrition, blood loss, and infectious diseases (e.g. malaria, hookworm, tuberculosis, HIV), among others. Anemia can cause delayed mental and physical development, fatigue, decreased work productivity, and increased risk of mortality, especially during childbirth. The condition is diagnosed by measuring the concentration of hemoglobin in the blood. Once the condition is diagnosed, the underlying cause can be determined and treated with, for example, iron supplements, anti-malarial drugs, or blood transfusions. As such, hemoglobin concentration assessment at the point-of-care enables clinicians to make rapid decisions about treatment resulting in improved medical care.

### SUMMARY

The present disclosure generally relates to systems and methods for diagnostic testing. More particularly, the present disclosure relates to system and methods for point of care diagnostic testing using signal transduction.

In one embodiment, the present disclosure provides a readout and signal transduction (ROST) component. The ROST includes a universal fixture adapted to receive and align one or more optical elements and one or more opto-mechanical elements, wherein the optical elements and one or more opto-mechanical elements are stacked in the universal fixture, one or more optical elements configured for integration into the universal fixture; and one or more opto-mechanical elements configured for integration into the universal fixture.

The present disclosure also provides a method for analyzing a sample. The method includes providing a readout and signal transduction (ROST) component, which includes a universal fixture adapted to receive and align one or more optical elements and one or more opto-mechanical elements, wherein the optical elements and one or more opto-mechanical elements are stacked in the universal fixture; one or more optical elements configured for integration into the universal fixture; one or more opto-mechanical elements configured for integration into the universal fixture; and a sample platform to accept and hold a sample for analysis. The method further includes putting an analyte on the sample platform and analyzing the analyte using the ROST component.

The features and advantages of the present invention will be apparent to those skilled in the art. While numerous changes may be made by those skilled in the art, such changes are within the spirit of the invention.

### BRIEF DESCRIPTION OF THE DRAWINGS

Some specific example embodiments of the disclosure may be understood by referring, in part, to the following description and the accompanying drawings.

FIG. 1 shows example ROST components according to the present disclosure.

FIG. 2 shows example optical and opto-mechanical elements.

FIG. 3 illustrates scattered light images for varying amounts of target DNA.

FIG. 4 illustrates scattered intensity in the red region for varying amounts of target DNA.

FIG. 5 illustrates immuno-fluorescence labeled cryptosporidium oocysts with miniature objective lens and with research-grade objective lens (Zeiss 40×/0.95).

FIG. 6 shows an example design of an interrogation unit of the present disclosure.

FIG. 7 illustrates optical elements, opto-mechanical elements, and images obtained with the optical elements.

FIG. 8 shows example elements for used in an interrogation unit in a ROST component.

FIG. 9 shows an example element configuration for interrogation units.

FIG. 10 shows an example optical multiplexing unit.

FIG. 11A shows an example ROST with references and sample interrogation units.

FIG. 11B illustrates a flow chart for signal processing.

FIG. 12A shows a photograph of an example portable reader for determining hemoglobin concentration from transmission measurements of blood spotted on chromatography paper.

FIG. 12B shows an example light path schematic for a portable hemoglobin concentration reader.

FIG. 13 shows an example extinction coefficient spectrum of unlysed blood on untreated Chr4 paper as calculated from collimated transmission measurement.

FIG. 14A shows an example graph of multiple spectrophotometric measurements on unlysed blood across a range of hemoglobin concentrations, where each line represents a set of experiments from a different day.

FIG. 14B shows an example graph of multiple spectrophotometric measurements on mechanically lysed blood across a range of hemoglobin concentrations, where each line represents a set of experiments from a different day.

FIG. 15A shows an example graph of calculated hemoglobin concentration over time for several samples using wavelengths of 528 nm and 656 nm.

FIG. 15B shows an example graph of calculated hemoglobin concentration over time for several samples using wavelengths of 508 nm and 656 nm.

FIG. 16 shows an example graph of calculated hemoglobin concentration from samples with different volumes of blood spotted on sodium deoxycholate-treated paper.

FIG. 17A shows an example graph of training (shown as diamonds) and validation (shown as asterisks) sets for the spectrometer.

FIG. 17B shows an example graph of training and validations sets for an embodiment of the present invention. The fit lines for graphs (A) and (B) are fit to the training set.

FIG. 17C shows an example Bland-Altman plot comparing hemoglobin values obtained from the HemoCue method to the values obtained for the validation set by the spectrometer.

FIG. 17D shows an example Bland-Altman plot comparing hemoglobin values obtained from the HemoCue method to the values obtained for the validation set by an embodiment of the present invention.

FIG. 18A shows an example graph of spectral measurement accuracy as compared to the HemoCue method.

FIG. 18B shows an example graph of percentage of hemoglobin measurements within ±1 g/dL of a corresponding HemoCue method result.

FIG. 19 shows an example schematic of an assembled embodiment of the invention and disassembled parts of an embodiment of the invention.

FIG. 20 shows an example exploded schematic of an embodiment of the invention.

FIG. 21A shows an example photograph of USB device for communicating with a computer system.

FIG. 21B shows an example filter lens that may be used in a ROST.

FIG. 21C shows an example objective lens that may be used in a ROST.

FIG. 21D shows an example LED that may be used for illumination in a ROST.

FIG. 21E shows an example detection component that may be used for illumination in a ROST.

FIG. 22 shows an example photograph of a wireless communication device that may be used to communicate and record results.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to systems and methods for diagnostic testing. More particularly, the present disclosure relates to system and methods for point of care diagnostic testing using signal transduction.

Certain implementations feature a high performance, low cost optical readout and signal transduction (ROST) component for point of care (POC) diagnostic assays, which is modular, robust, reconfigurable, and highly scalable in volume. Certain example ROST include a universal fixture designed to accept interrogation units for illumination and light collection which can operate in multiple measurement modes, including transmission, reflectance, scattering, and fluorescence, in a single spectral band or in multiple bands.

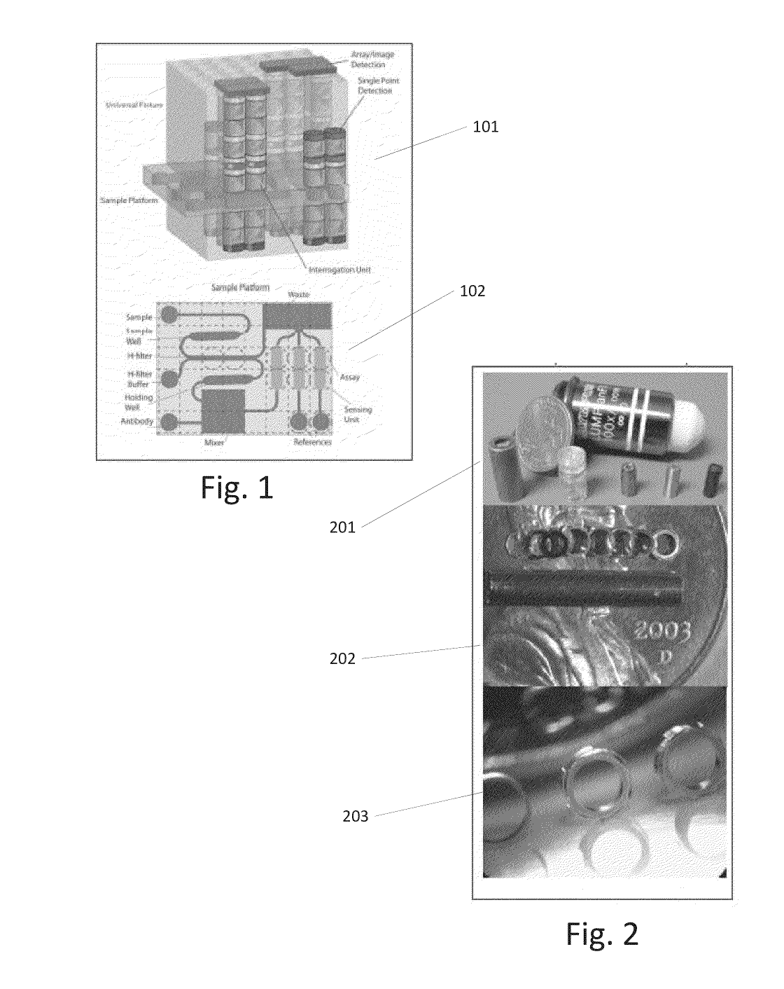

FIG. 1 shows two views of an example ROST. The example ROST 101 shown in FIG. 1 includes a universal fixture, a sample platform, an integration unit, a single point detection unit, and an array image detection unit. The example ROST 101 illustrates the universal fixture coupled to a hypothetical microfluidic sample platform. Interrogation units provide illumination and detect signal.

The schematic view 102 of the example ROST shown in FIG. 1 includes a sample that is connected with a sample well, an H-filter, an H-filter buffer, a holding well, and antibody, a mixer, references, a sensing unit, an assay, and a waste unit. The schematic view 102 illustrates interrogation points (circles) which can be used to monitor fluid flow, mixing, as well as sample and reference points in multiple measurement modes.

In certain implementations the interrogation units are assembled from a standardized library of optical and opto-mechanical elements that stack inside the fixture without alignment. The fixture is configured to accept interrogation units arranged in any pattern, permitting easy customization for a wide variety of existing and new sample platforms without the need for complicated and expensive redesign.

Certain implementations feature optical readout and signal transduction (ROST) components for POC diagnostic assays. The ROST component consists of a universal fixture designed to accept interrogation units for illumination and light collection which can operate in multiple measurement modes, including transmission, reflectance, scattering, and fluorescence, in a single spectral band or in multiple bands. The ROST component is highly modular. Interrogation units are assembled from a standardized library of optical and opto-mechanical elements that stack inside the fixture without alignment. The fixture is designed to accept interrogation units arranged in any pattern, permitting customization for a wide variety of existing and new sample platforms without the need for complicated and expensive redesign.

Certain implementations use semiconductor-based light sources and digital CMOS array detectors, harnessing their increasing performance levels, shrinking footprints, and falling costs in the consumer market-driven sector of the opto-electronics industry. Example implementations include one or more mass-fabricated, small-scale, optical and opto-mechanical elements (FIG. 2).

Example implementations may be used in the readout stage of POC diagnostic tests. Example ROSTs are applicable for all assays with optical readout. The universal fixture is designed for integration with a variety of assay platforms, including paper, plastic, and glass. The ROST component is adaptable to a diverse range of targets (e.g. metabolite, protein, nucleic acid, organism) and sample types (e.g. stool, serum, saliva). Example interrogation units are for a variety of measurement modes, including transmission, reflectance, light scattering and fluorescence. Example components allow the incorporation of polarization-sensitive and multispectral analysis within these modes. Interrogation units can operate at multiple spatial resolution scales, ranging from diffraction-limited imaging to simple spatial integration of signal across an entire measurement channel. These modes provide the ability to detect and quantify commonly used optical labels, including colorimetric dyes, polystyrene microspheres loaded with absorbing or fluorescent dyes, fluorescent dyes and molecular beacons, metallic nanoparticles, and semiconductor nanocrystals. As shown in FIG. 1, the universal fixture provides the flexibility to interrogate the assay platform at multiple points to quantify regions which may contain unlabeled specimen (background) or other reference channels to aid in calibration/quantification. The ability to detect spectral signatures can provide multiplexing capability, as well as to separate signal from labeled targets (which have well-defined spectral signatures) and interferrants (which often produce broad spectral background). The ability to obtain diffraction-limited images from regions of interest on the sample platform provides further ability to discriminate between target and background based on morphology of labeling.

The sensitivity of example ROST is primarily dependent on the signal-to-noise ratio and the signal-to-background ratio which can be achieved. While these depend on the choice of label, amount of target present, degree of background interferrant, system throughput, and detector characteristics, example models quantitatively analyze optical throughput and relate this to limits of detection for particular assay conditions. One example optical label is an oligonucleotide-targeted gold nanoparticles to detect the presence of cryptosporidium in stool samples.

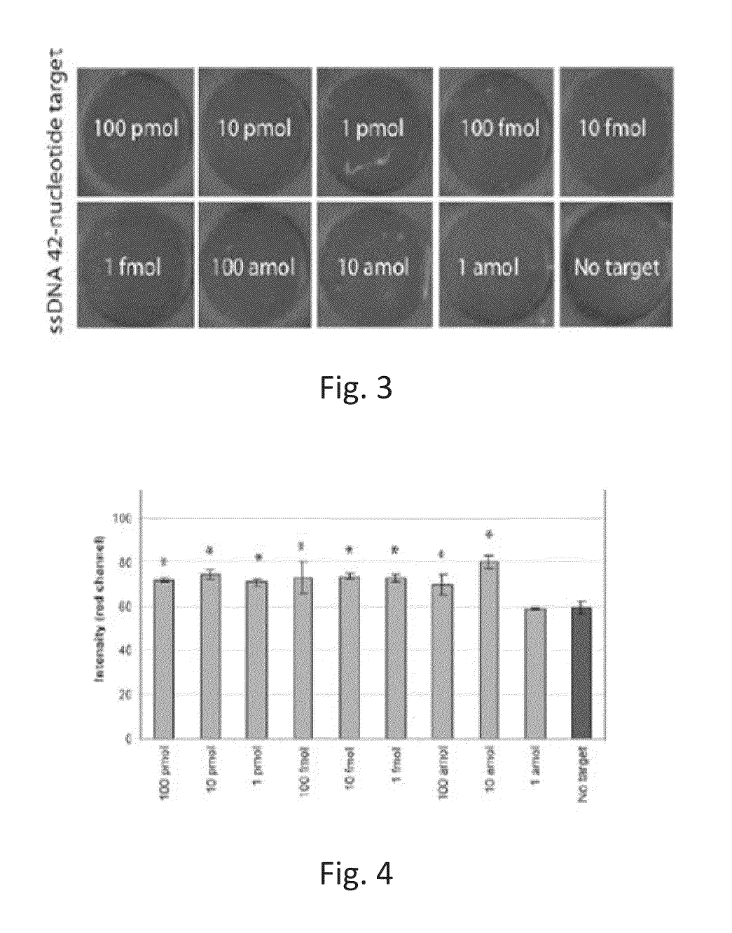

FIGS. 3-4 illustrate the aggregation and resulting color shift in the light scattering of the nanoparticles in the presence of complementary target DNA. FIG. 3 illustrates scattered light images while FIG. 4 illustrates scattered intensity in the red region for varying amounts of DNA target labeled with gold nanoparticles conjugated to complementary oligonucleotides. The presence of target induces nanoparticle aggregation, causing a red shift in the scattering. In this modality, as little as 10 amol (10⁶ copies) of target is the limit of detection in darkfield imaging. Using spectroscopic interrogation may improve the limit of detection by one additional order of magnitude and provides the ability to quantitate the amount of target present. An example ROST can detect this signal in both measurement modes.

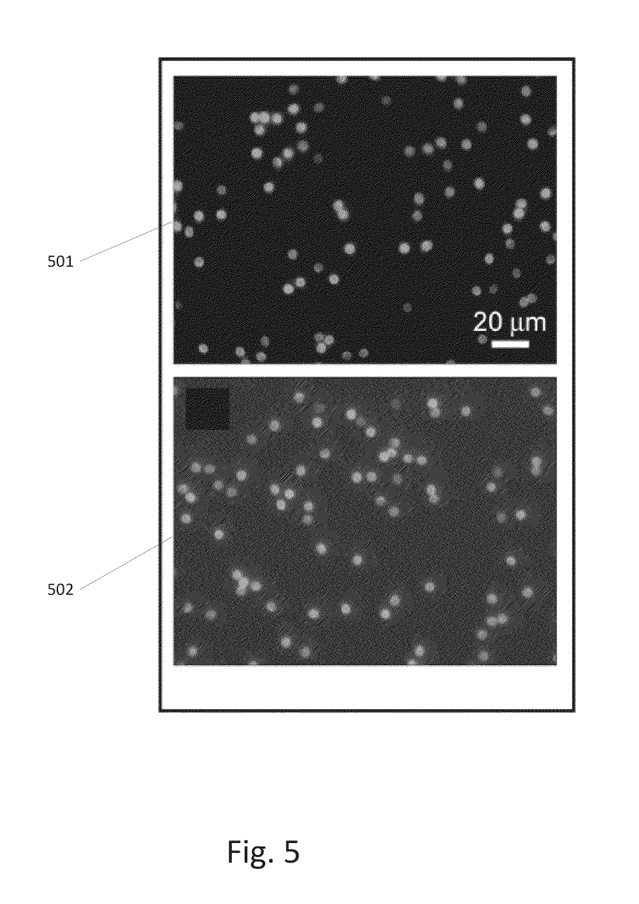

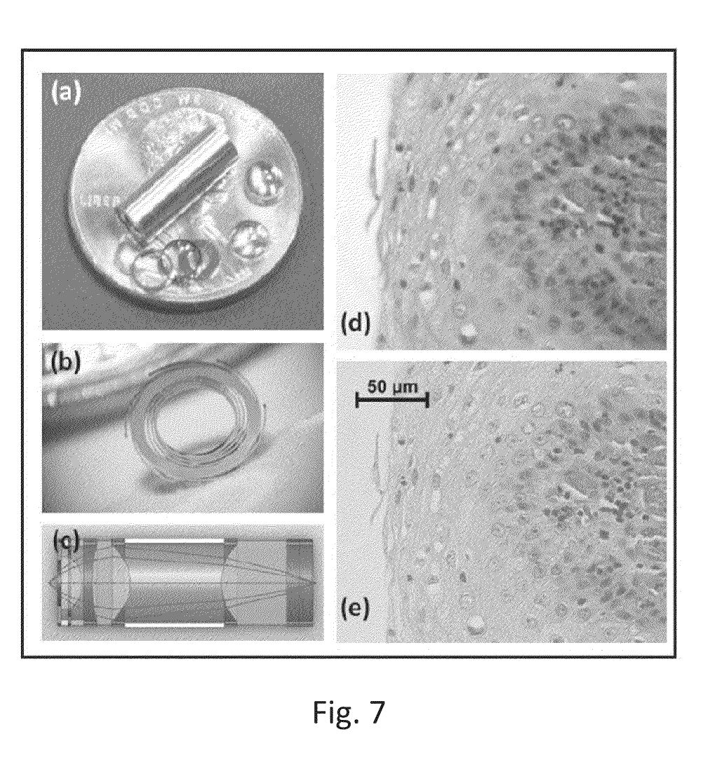

In the example of FIGS. 3-4, a traditional large-scale optical microscope was used to obtain images of scattered light. We have also used the high performance low-cost miniature optical systems to interrogate labeled sample, demonstrating the ability to achieve optical performance comparable to macroscopic systems with a reduction in size and cost of more than two orders of magnitude. FIG. 5 shows fluorescence images of cryptosporidium oocysts labeled with an antibody-targeted fluorescent dye. The bottom image 502 was obtained with a research-grade fluorescence microscope (Zeiss 40×/0.95), while the top image 501 was obtained using a miniature optical system fabricated using the zero-alignment concept of the present disclosure (shown in FIG. 7). In certain implementations, an advantage of the proposed technology is the ability to design and fabricate simple systems for image-based sensing. Certain implementation feature a palm-sized, reusable ROST component which can be produced for <$10 and can easily be integrated with a variety of assay platforms. In certain implementations, the device can operate continuously for 8-10 hours on a single battery charge.

In certain implementations, the optical elements are held in pace using opto-mechanical mounts which are made using a lithographic process. For example, the opto-mechanical components may be fabricated with X-ray (DXRL) or UV-lithography. In certain implementations, the universal fixture is made using injection molding.

Low-cost, mass-production methods are capable of generating high-quality miniature-scale optical elements as evidenced by the ubiquitous presence of cameras in cell phones, and other portable electronic devices. We have shown that the same methods are also capable of generating relatively more challenging microscope optics for commercial products. Certain implementations of the present disclosure may yield a new, modular design approach to build a reusable ROST component that is more sensitive, robust, compact, power efficient, and cheaper than large-scale counterparts and that can easily be customized to interface with a wide variety of POC sample platforms. An exemplary ROST component is based on a universal fixture to accommodate interrogation units in an arbitrary pattern is shown in FIG. 1.

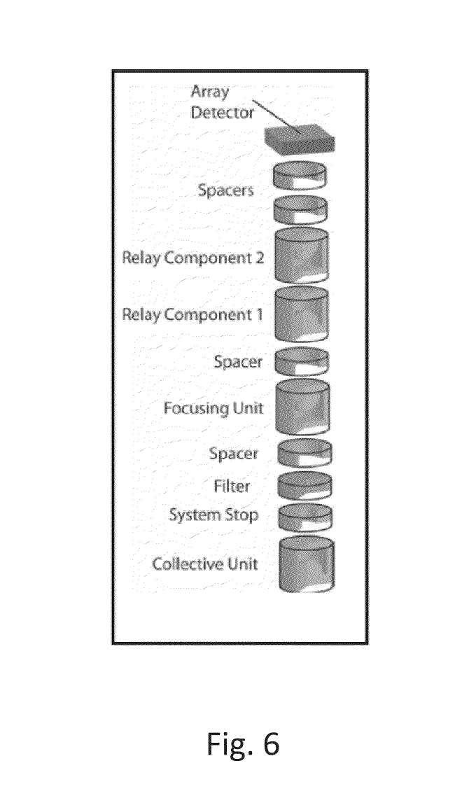

In certain example implementations, each interrogation unit is comprised of standardized optical elements and miniature opto-mechanical mounting elements which are stacked within tubes in the fixture as shown in FIG. 6. The ROST of FIG. 6 includes an array detector, a first spacer, and second spacer, a relay component 2, a relay component 1, a third spacer, a focusing unit, a fourth spacer, a filter, a system stop, and a collective unit. Other implementations may include fewer, more, or different elements. The ROST of FIG. 6 may be referred to as a generalized design of a “plug & sense” interrogation unit. Example interrogation units include optical elements, one more spacers, one or more relays, one more sources, and one or more detectors. In example embodiments, elements are stacked in a universal fixture with opto-mechanical mounts to self-align the system.

In certain implementations, all components are mounted inside the fixture using a proven zero-alignment microscopic optical-system concept. In practical terms, the zero-alignment concept translates into assembly errors that are smaller than the tolerances on the performance of the optical system. No, or minimal, adjustment is needed and assembly works in a “plug & sense” fashion. Certain implementations of the present disclosure, rely on two advanced manufacturing techniques to fabricate high-performance, low-cost “plug & sense” interrogation units. Example miniature plastic optical elements will be manufactured using injection molding. An advantage of injection molding is that it permits the use of aspheric surfaces, requiring fewer lens elements in an interrogation unit for the optimum optical quality. Moreover, injection molding may allow for extremely low per-element costs at high volume. Diamond turning can be used to rapidly fabricate prototype elements in low volume at reasonable cost and, once perfected, molds for injection molding of such parts. Just as lens elements can be miniaturized, so can optomechanical mounts. Certain implementations employ ultra-precise deep X-ray or UV lithography to manufacture such mounts in a batch mode as shown in FIG. 7.

FIG. 7 illustrates a photo of a 3.9 mm diameter, NA 1.0 objective alongside optical elements and opto-mechanical mounts (a), a self-centering opto-mechanical mount which consists of a series of springs (b), a layout of objective (c), a transmitted light image of H&E stained buccal mucosa, acquired with the objective in part a (d), and an image of the same tissue acquired on a research-grade scope (Zeiss Z.1, 40×/0.95) (e).

Example optomechanical mounts can include precision features such as external and internal springs to compensate for variations in the outer diameter of optical elements or the

TABLE 4
Optical and opto-mechanical elements and manufacturing technologies

| Functional Parameter | Optical Elements | Manufacturing Tech. | Opto-Mechanical Elements | Manufacturing Tech. |
|---|---|---|---|---|
| Numerical Aperture | Lenses | Injection molding | Self-centering springs | Injection molding |
| Optical Power | Relay components | Glass press molding | Kinematic components | Stamping |
| Throughput | Prisms | Stamping | Spacers | DXRL |
| Spectral Resoluton | Gratings | CNC Diamand Machining | Mounting barrels | UV Lithography |
| Magnification | Filter plates | Ruling | | |
| Detection principle | Beam splitters | Dicing | | |
| | Pinhole Grids | Multilayer coating | | |

inner diameter of slots in the universal fixture, and self-center multiple elements. Table 4 shows example functional parameters of the ROST component, as well as example optical elements and optomechanical elements and example manufacturing technologies which may be used to manufacture the optical and opto-mechanical elements.

TABLE 5
Candidate materials for reference standards & relevant concentrations from the literature

| | | | |
|---|---|---|---|
| | | Biomedically Relevant | |
| Reference Material | Example Target Assay | Concentration Range (LOD) | Ref. |

Absorbance

| | | | |
|---|---|---|---|
| Glucose | Glucose (metabolite) in paper microfluidic | 0-550 mM (0.5 mM) in 0.5 μL | [32] |
| oxidase/HRP | | reaction volume | |
| Tetrabromophenol | Bovine Serum Albumin (protein) in paper | 0-40 μM (4 μM) in 5 μL | [32, 33] |
| Blue (TBPB) | microfluidic | reaction volume | |
| Dyed microspheres | Giemsa assay (Plasmodium sp. stain) | ≧50 beads/μl in 50 μl volume | [34] |
| | Ziehl-Neelsen assay (M. tuberculosis stain) | ≧1 bead in 20 μl volume | [35] |
| | Bacillus anthracis (RNA) in lateral flow | 8 × 10⁹ dyed microspheres in | [27] |
| | microarray | 10 μl volume | |

Scattering

| | | | |
|---|---|---|---|
| Gold nanoparticles | S. aureus (DNA) | 10-40 pM in 15 μl reaction | [36] |
| (50 nm) | Cryptosporidium (DNA/RNA) | volume | [31] |

Fluorescence

| | | | |
|---|---|---|---|
| Fluorescent MESF | Acridine orange and DNA intercalating | 1-4 beads/μl in 50 μl volume | [34, 37, |
| microspheres | dyes (staining P. falciparum); Auramine- | | 38] |
| | Rhodamine (staining M. Tuberculosis) | | |
| Fluorescent DNA | FAM-labeled primers for in-chip PCR | 200 nM ssDNA in 200 nL | [39] |
| oligonucleotides | | reaction volume | |
| | dsDNA labeled with SYBR-green/YOYO- | 1-10 μM dye-labeled dsDNA | [40, 41] |
| | 1/YOPRO DNA-intercalating dyes | in 550 nL reaction volume | |
| | in-chip detection of amplified DNA using | 2 μM molecular beacon in 1 | [42] |
| | molecular beacons | μl reaction volume | |

In certain implementations, biomedically relevant reference standards are used to test ROST components. Table 5 lists example candidate materials for each modality as well as references citing relevant concentration levels for biomedically relevant targets.

Certain implementations include one or more interrogation units for illumination, transmission, reflectance, light scattering, and fluorescence modes. Certain implementations include one or more optical multiplexers. Example interrogation units are for generation and collection of optical signals in several modalities, including, but not limited to transmission, reflectance, light scattering, and fluorescence modes. Optical multiplexers are components that will permit parallel illumination and signal collection from multiple distributed regions of a diagnostic test. Interrogation units and optical multiplexers will be fabricated within this objective and tested for optical performance at the individual component level using recognized optical standards.

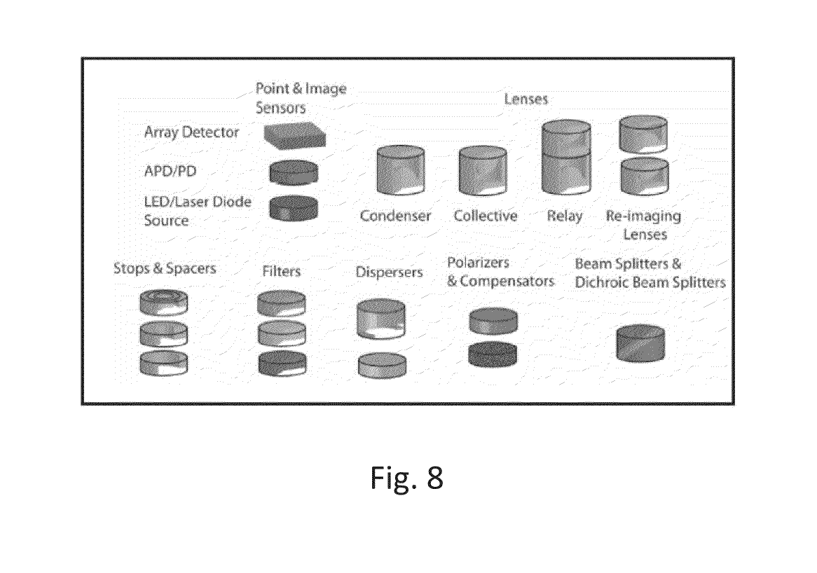

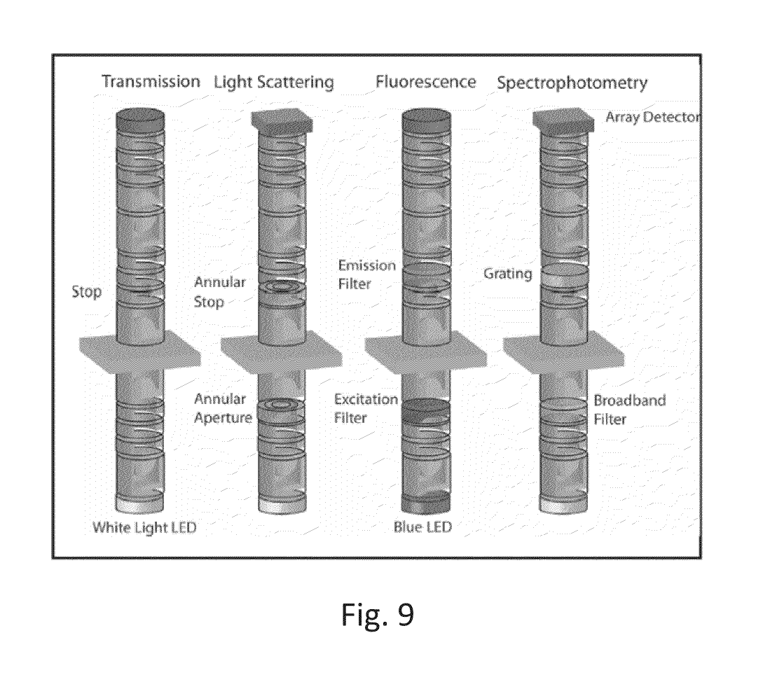

Example interrogation units for the ROST component may originate from a base catalog of individual components, allowing for distinct interrogation units to be developed in a cost-efficient process. FIG. 8 shows examples of these individual elements used to configure interrogation units in the ROST component, with FIG. 9 illustrating their assembly into complete interrogation units for transmission, reflectance and fluorescence modes. As shown in FIG. 8, example ROST units may include zero, one, or more of each of: array detector, avalanche photodiode (APD), photodiode, lenses, stops, spacers, filter, dispersers, polarizers, compensators, beam splitters, and dichroic beam splitters. Example lenses are configured to be used as condensers, collectives, relays, and re-imaging lenses. In addition, FIG. 9 illustrates an example interrogation unit with the addition of multispectral analysis, including examples of elements to configure interrogation units for different detection modes. As shown in FIG. 9, these include (from left to right), interrogation units for transmission, light scattering, fluorescence, and transmission with spectroscopic sensitivity.

The design parameters for individual components (for example, lens diameter, thickness, and curvature) may be based on the needs of the system and its application. In certain implementations, tolerances for interrogation units for nonimaging-based diagnostic tests (those purely requiring efficient light collection) may be less stringent than for the high-resolution imaging previously demonstrated (FIGS. 2, 5 & 7).

The highly modular approach of the ROST component may be extended to the individual component level (FIG. 8), whereby each will be designed to meet the requirements of the zero-alignment assembly technique (FIG. 7). This will allow example interrogation units (FIG. 9) to provide a range of operating parameters including magnification, numerical aperture, spectral range etc., to be assembled under a common process. In some example implementations, the functionality of interrogation units can be changed; for example, switching from transmission to light scattering to fluorescence mode by changing of only stops, spacers, and filters.

Example interrogation units are for sample illumination and optical signal collection from a single region within a diagnostic test. (The test may be a lateral flow strip, a microfluidic cartridge, or other format, but the diagnostically relevant information is confined to a single spatial region). Example individual lens elements (FIG. 7) are for collecting, condensing, relaying, and imaging light. Example interrogation units including one or more stops, spacers, filters, polarizers, or dispersive elements. Example interrogation units perform illumination and detection in transmission mode (FIG. 9 left). Other example interrogation units employ one or more of light scattering, fluorescence, and spectrograph.

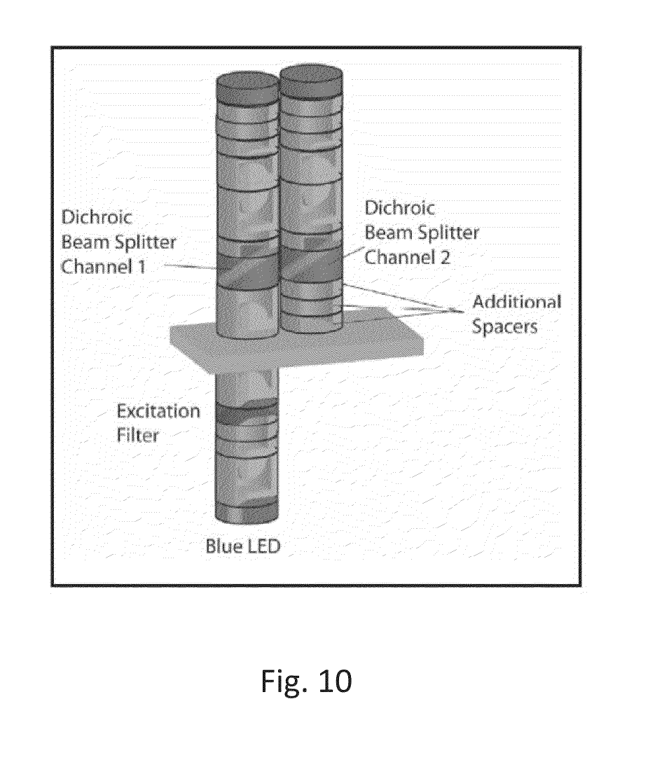

Example ROST elements are for multiplexed analysis. In these elements, optical multiplexers enable simultaneous evaluation of multiple regions within a single diagnostic test. (These elements could be applied to evaluate discrete capture lines on a lateral flow strip, or separate chambers in a microfluidic cartridge, for example). Example optical multiplexers enable simultaneous illumination of multiple test regions with a single optical light source. Other example optical multiplexers enable simultaneous collection of optical signals from multiple test regions for delivery to a single detector. FIG. 10 illustrates an example of an optical multiplexing unit enabling simultaneous analysis of multiple analytes with different fluorescence emission.

Example implementations of the present disclosure further use signal processing tools for detection in multiple modalities. In certain implementations a step-wise strategy is used to design signal processing modules that can be combined in flexible ways to yield desired output signals and to characterize signal-to-noise levels, signal-to-background ratios, minimum assay detection limits, assay linearity, and assay dynamic range.

Development may include defining the type of signal to be measured; identifying sources of variation in signal associated with both target and background; and developing analog/digital processing tools to yield calibrated signals. In certain implementations, the desired output signal type for each measurement mode is determined. Using suitable detectors, experimental testing may be performed using single-channel interrogation units to identify necessary calibration signals to be collected, including, but not limited to: source intensity/power, ambient light levels, and system throughput. The process may further include developing signal processing strategies to extract desired output signals, calibrated for environmental variations. An approach based on a single interrogation channel, is straightforward, and enables a user to characterize the sensitivity, specificity, and dynamic range of each modality. In other implementations, by combining information from multiple channels, internal control channels may be integrated to better compensate for environmental variations and for the presence of interferrants.

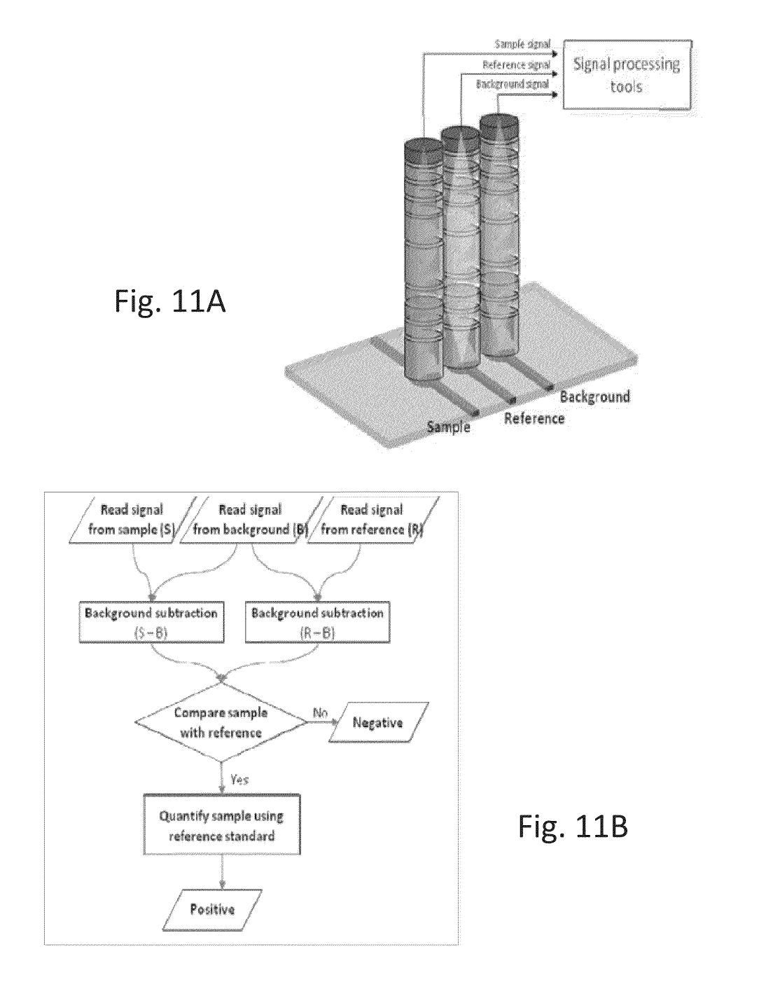

In certain implementations ROST calibration units are included in the universal fixture. FIG. 11A shows a simple example, incorporating signal, reference, and background interrogation units. While this example shows a single sample channel, additional channels can easily be added and combined within the universal fixture. FIG. 11B illustrates a flowchart for an initial approach to signal processing designed to combine outputs to a meaningful output voltage for this configuration. More complex tools can be developed if needed to achieve desired sensitivity and specificity, for example incorporating variations in illumination intensity or lock-in detection.

Example implementations include a Single field of view ROST where optical illumination and signal collection is performed from a single site on the assay; for example a color indicator bar on a lateral flow test. Another example implementation is a distributed field of view ROST where optical illumination and signal collection is from multiple sites on the assay; for example reading fluorescence from multiple chambers of a microfluidic card. Another example implementation is a continuous field of view ROST where optical illumination and signal collection is from a near continuous region of the assay; for example examination of brightfield or fluorescence images across a large region of a microfluidic card or a malaria or tuberculosis smear.

It is noted that embodiments of the present invention may be used for both imaging and non-imaging applications. Some examples of the non-imaging applications contemplated for the invention include hemoglobinometers, colorimetric assays, and Fluorescence assays. Some examples of imaging applications of the invention relate to imaging biological samples and bead based assays. It is appreciated that there may be many other applications for the present invention.

To facilitate a better understanding of the present disclosure, the following examples of certain aspects of some embodiments are given. In no way should the following examples be read to limit, or define, the entire scope of the disclosure.

### EXAMPLES

Chromatography paper may be a low-cost, rugged, and self-contained medium for microfluidic assays in point-of-care systems suitable for developing countries. The ubiquitous and low-cost nature of chromatography paper represents an alternative to expensive plastic cuvettes used in other systems such as HemoCue. An objective of this example is to investigate if chromatography paper can serve as a low-cost medium for accurate spectrophotometric detection of blood hemoglobin concentration.

In order to validate a paper-based spectrophotometric hemoglobin assessment, we performed experiments to optimize or evaluate sensitivity to five parameters: impregnating paper with chemicals to lyse red blood cells, paper type, drying time, wavelengths measured, and volume of blood. Blood and plasma samples were obtained via venous draw from healthy donors who gave informed consent; plasma was also purchased from the Gulf Coast Regional Blood Bank (Houston, Tex.). Protocols were reviewed and approved by the Institutional Review Board at Rice University. For these experiments, whole blood was diluted with plasma to simulate anemia. Micropipettes were used to spot blood on paper to simulate touching a patient's fingertip to the paper after a fingerprick. A benchtop spectrophotometer (Cary 5000 UV-VIS) recorded the spectra of the samples via an on-axis collimated transmission measurement. The samples were masked with a 3 mm×3 mm aperture. Gehring et al. have reported the HemoCue 201+ to be accurate to within approximately 0.5 g/dL of the reference method in a laboratory setting (H. Gehring, et al., Acta Anaesthesiologica Scandinavica, 2002, 46, 980-6.), so it was used as a standard for the evaluation of the chromatography paper medium. Values from the spectrum of blood spotted on paper were correlated with the hemoglobin concentration of the sample obtained with a HemoCue.

Seven filter and chromatography papers from Whatman PLC (United Kingdom)—chromatography papers Chr1, Chr3MM, Chr4, and grades 2, 4, 5, and 6 filter paper—were evaluated for (1) the qualitative appearance of uniform spreading of blood spotted on the paper and (2) the repeatability of spectrophotometric measurements of blood samples with high and low hemoglobin concentrations. Paper strips were treated with 10 μL 4% (w/v) sodium deoxycholate in PBS and allowed to dry. 10 μL of blood was applied and spectra were taken 2 minutes after spotting. For each type of paper, three measurements were made of blood with a low hemoglobin concentration (7-8 g/dL) and three of blood with a high hemoglobin concentration (15-16 g/dL). The measurements of Chr4 had the lowest standard deviation for both hemoglobin concentrations for papers where blood spread quickly and with little pooling. Chr4 was used for all other experiments.

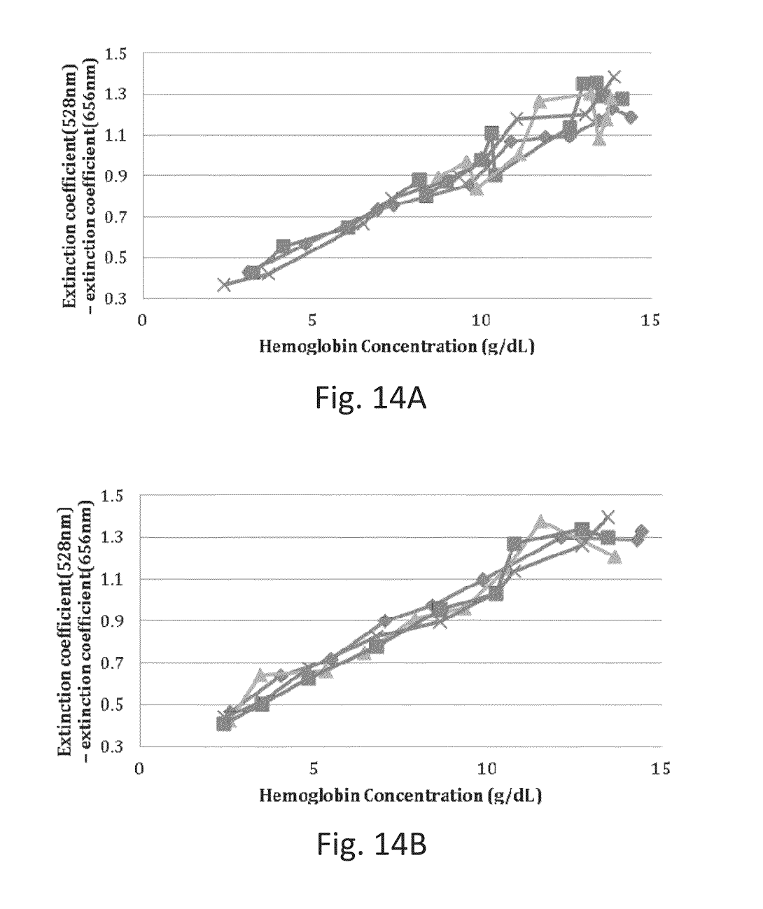

We examined whether hemolysis affected the variability of light transmitted through paper spotted with blood. In general, blood spread more uniformly and transmission measurements from unlysed blood (FIG. 14A) showed greater variability than mechanically lysed blood (FIG. 14B), particularly at hemoglobin concentrations exceeding 10 g/dL. The repeatability of transmission measurements of blood on sodium deoxycholate-treated paper was similar to that of mechanically lysed blood on untreated paper.

We examined whole blood samples as well as samples where red blood cells were mechanically lysed or chemically lysed with sodium deoxycholate. To achieve mechanical lysis, whole blood was taken through multiple (>3) freeze-thaw cycles (−20° C. to 20° C.). The blood was considered lysed if high-speed centrifugation did not separate the blood into plasma and red blood cell layers and microscopy showed no intact cells.

To achieve chemical lysis of whole blood spotted on paper, we used the detergent sodium deoxycholate. To evaluate the amount of sodium deoxycholate needed to achieve lysis on paper, paper strips were treated with various volumes (10, 20, 30, 40, and 50 μL) of 2% or 4% (w/v) sodium deoxycholate in PBS. After the sodium deoxycholate dried, 10 μL whole blood was applied to the treated paper. Unlysed blood and mechanically lysed blood applied to untreated paper served as controls. The blood dried for 2 minutes and was then eluted from the paper in 1 mL PBS for 10 minutes. The paper was removed and the spectrum of the remaining solution was taken on a Cary 5000 UV/VIS spectrophotometer from 350 nm to 800 nm. Blood was considered lysed if its transmission spectra did not show evidence of the turbidity associated with intact red blood cells. 10 μL of 4% (w/v) sodium deoxycholate was chosen for all further experiments because (1) it resulted in effective lysis and (2) the blood spread quickly and evenly on the paper without pooling.

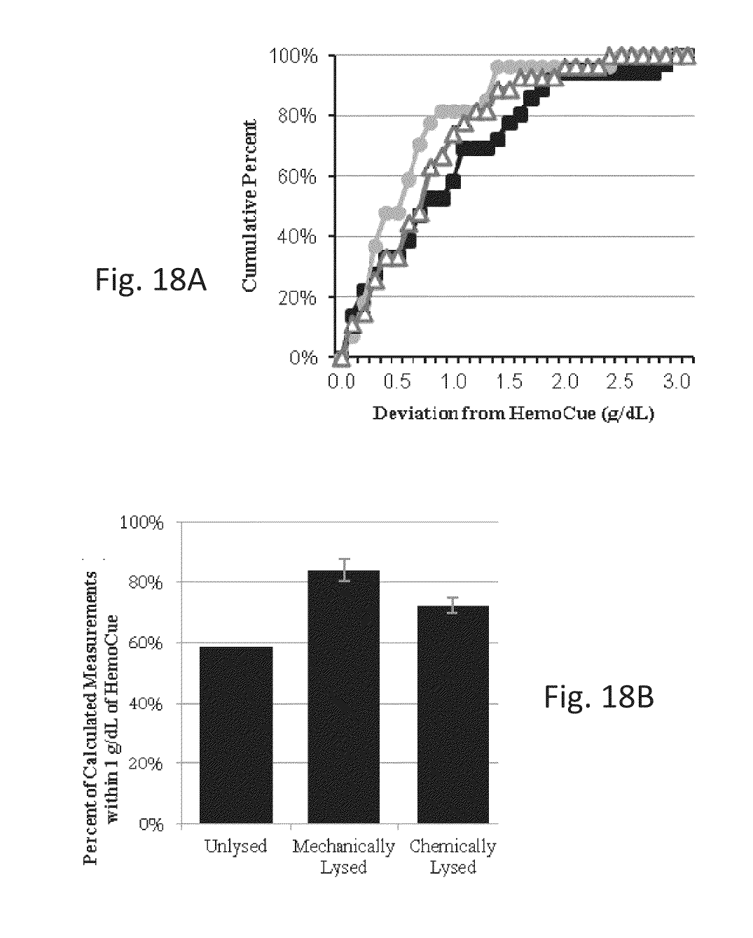

To assess the effect of red blood cell lysis on the accuracy of hemoglobin concentration derived from spectral measurements, we measured transmission spectra of samples with varying concentrations of hemoglobin for three conditions: unlysed blood applied to untreated Chr4 paper, mechanically lysed blood applied to untreated Chr4 paper, and unlysed blood applied to sodium deoxycholate-treated Chr4 paper. Blood from 4 donors was diluted with plasma to obtain a range of hemoglobin concentrations spanning the physiologic range. The experiment was repeated on two days for each condition; half of the data was used as a training set, and the remaining data were used as a validation set. The training set for each condition was used to develop an algorithm relating hemoglobin concentration as measured by HemoCue to the absorbance difference at two wavelengths as described herein. This relationship was used to calculate the hemoglobin concentration of samples in each validation set, and the calculated hemoglobin concentrations were compared to the concentrations obtained from HemoCue. FIG. 18A shows the cumulative percentage of samples with a given deviation from HemoCue where unlysed blood are represented as squares, mechanically lysed blood as circles, and chemically lysed blood as triangles. The deviations from HemoCue were binned in 0.1 g/dL increments, and the cumulative percentage of samples with a given deviation was plotted for the three conditions.

FIG. 18B shows the percent of samples for a given condition that are within ±1 g/dL of the hemoglobin concentration obtained by HemoCue. The highest percentage of samples was within ±1 g/dL when using mechanically lysed blood. On average, accuracy was slightly reduced when using chemically lysed blood. Accuracy was lowest when using unlysed blood. Though chemical lysis with sodium deoxycholate does not give the level of accuracy achieved with mechanically lysis, it represents an improvement over unlysed blood, and chemical lysis with pre-treated paper can be easily performed at the point of care.

To determine the effects of drying time and to select optimal wavelengths for determining hemoglobin concentration, absorption spectra of whole blood spotted on sodium deoxycholate-treated Chr4 paper were collected from 350 nm to 800 nm over the course of 30 minutes, the time during which a hemoglobin concentration assessment could reasonably be performed in the field. Blood samples from four donors were used, and spectra were taken every 2 minutes. This data helped determine which spectral measurements could be used to calculate consistent hemoglobin concentrations even if the drying time of the spot varied, an important factor for point-of-care use.

The difference in optical density between various pairs of wavelengths was plotted versus time, and a training set, an example of which is described herein, was used to calculate hemoglobin concentration. The algorithm was derived from training set spectra taken 2 minute after spotting; this algorithm was used to calculate hemoglobin concentration at each time point.

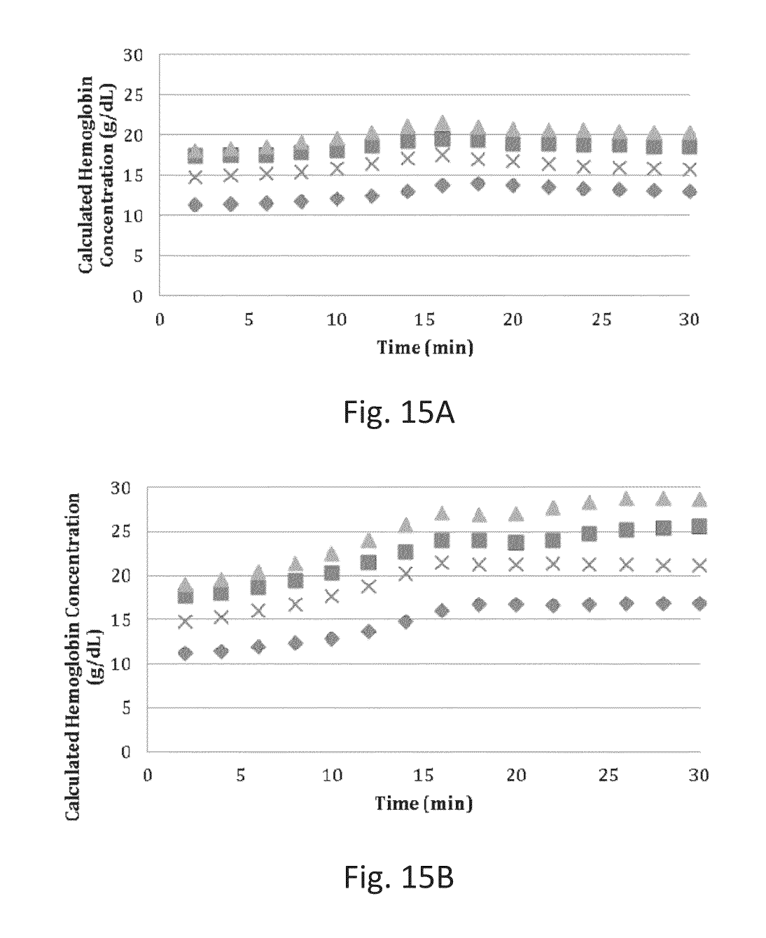

Some pairs of wavelengths may give results that are stable over time, such as the difference between 528 nm and 656 nm (an average increase of 1.5 g/dL over 30 min., FIG. 15A). Other pairs, such as 508 nm and 656 nm, may result in increasing hemoglobin concentrations over time (an average of 7.4 g/dL, FIG. 15B). The choice of wavelength in the region where hemoglobin does not absorb had little effect on the results. Other choices of wavelengths may also give consistent results over time, but 528 nm and 656 nm were chosen in this example to match the availability of LEDs for our low-cost reader and are not intended to limit the current invention.

For the remainder of the experiments, spectra were taken at 2 minute after spotting and the absorbance difference between 528 nm and 656 nm was used to calculate hemoglobin concentration and compare to results determined by HemoCue.

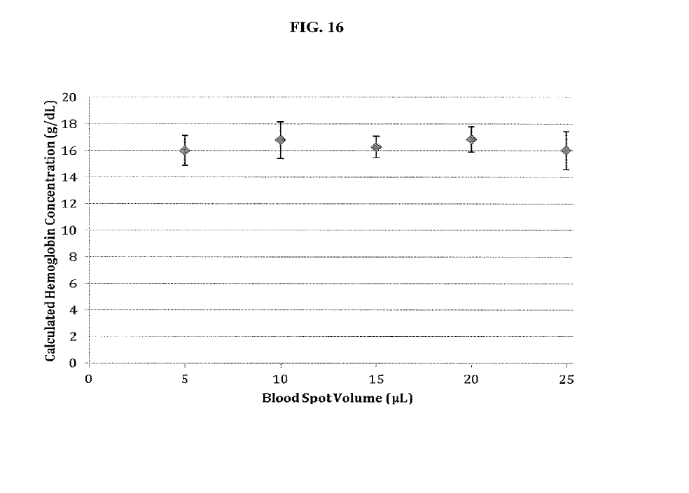

Because the volume of blood in a finger-prick can vary, we examined how sensitive our method was to changes in blood spot volume. Absorption spectra of various volumes (5, 10, 15, 20, 25 μL) of whole blood on sodium deoxycholate-treated Chr4 paper were obtained to determine the effects of blood volume on the test. (FIG. 16) Three measurements were taken for each volume. Transmission spectra were used to calculate hemoglobin concentrations based on a training set of samples from 20 patients.

The calculated hemoglobin concentrations for all volumes tested (5 μL to 25 μL) are not statistically different (p=0.82), so this range of volumes is considered appropriate for measurements. A volume of 2.5 μL was also evaluated, but this volume of blood was not large enough to fill the 3 mm×3 mm aperture used in the spectrometer and thus gave highly variable results.

To help health workers gauge that a sufficiently large volume has been spotted on the paper, eight small dots may be laser printed onto the paper in the shape of a circle. This circle may hold roughly 10 μL of blood. This circle may also denote where the sodium deoxycholate lysing agent has been dried onto the paper.

Hemoglobin concentration from on-axis transmission measurement of blood spotted on paper were calculated. Patient blood samples were used to train and validate this algorithm on the laboratory spectrometer. Whole blood samples were obtained from 43 hospitalized patients. Venous blood was collected in heparinized tubes; anonymous specimens were obtained one week after collection. 3 patient samples were discarded because the blood showed significant clotting. Data from 20 samples were used to develop an algorithm relating the hemoglobin concentration as measured by HemoCue to the absorbance difference between two wavelengths using a best-fit power curve: [Hb] in g/dL=A*[Extinction coefficient(λ₁)−extinction coefficient(λ₂)]ⁿ, where A, λ₁, λ₂, and n were varied.

Patient blood samples were used to train and validate this algorithm on the laboratory spectrometer. Whole blood samples were obtained from 43 hospitalized patients. Venous blood was collected in heparinized tubes; anonymous specimens were obtained one week after collection. 3 patient samples were discarded because the blood showed significant clotting.

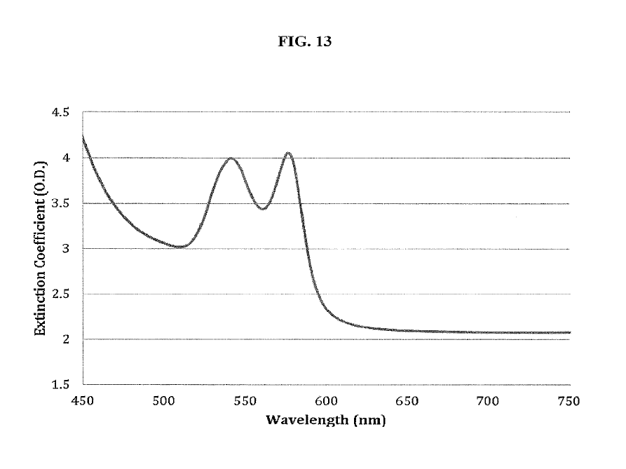

Two wavelengths are used: a hemoglobin absorption range from 350 nm to 600 nm where hemoglobin absorbs and a control absorption range from 600 nm to 750 nm where hemoglobin does not absorb. From 350 nm to 600 nm, spectra of blood on paper show characteristic absorbance peaks due to hemoglobin; above 600 nm, spectra show a nearly flat baseline region due to the paper substrate (FIG. 13).

In one embodiment, data from 20 samples were used determine the absorbance difference between 528 nm and 656 nm to the hemoglobin concentration as measured by HemoCue using a best-fit power curve. Data from 20 samples were used to develop an algorithm relating the hemoglobin concentration as measured by HemoCue to the absorbance difference between two wavelengths using a best-fit power curve.

In one embodiment, the pair of wavelengths used were 528 nm and 656 nm. Data from the remaining 20 samples were used to validate the performance of this algorithm. The hemoglobin concentration for these samples was calculated from the transmission data using the relationship from the training set; results were comparable to those measured by the HemoCue. For both training and validation sets, a reading was taken on the spectrometer at 120 sec. after spotting.

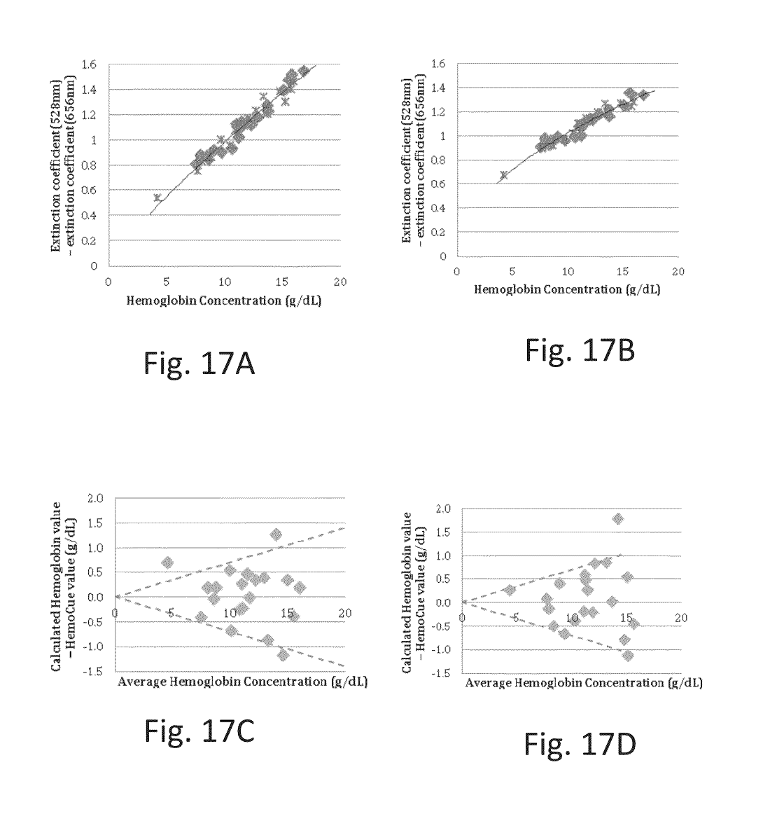

Results from the training and validation sets for both the spectrometer and the low-cost reader are shown in FIGS. 17A-17B. The Bland-Altman plots (FIGS. 17C-17D) show that both devices are accurate to within 1 g/dL of the HemoCue device for 95% of samples. Gomez-Simon et al. determined that HemoCue values are accurate to within approximately 1.5 g/dL of the hemoglobin concentration determined by a laboratory hematology analyzer.

The WHO hemoglobin color scale also uses blood spotted on paper to determine hemoglobin concentration. With this method, a blood spot is compared to reference standards ranging from 4 g/dL to 14 g/dL in gradations of 2 g/dL. In non-optimal conditions such as poor lighting or inadequate training, however, accuracy can drop to ±4 g/dL. Van den Broek et al. reported that the color scale was within 2 g/dL of a laboratory hemoglobin value in only 67% of cases. (van den Broek, et al., Diagnosing anaemia in pregnancy in rural clinics: assessing the potential of the Haemoglobin Colour Scale. Bulletin of the World Health Organization. 1999; 77(1):15-21.) Thus, our spectrophotometric method has the potential to provide increased accuracy over the current paper-based method with comparable cost.

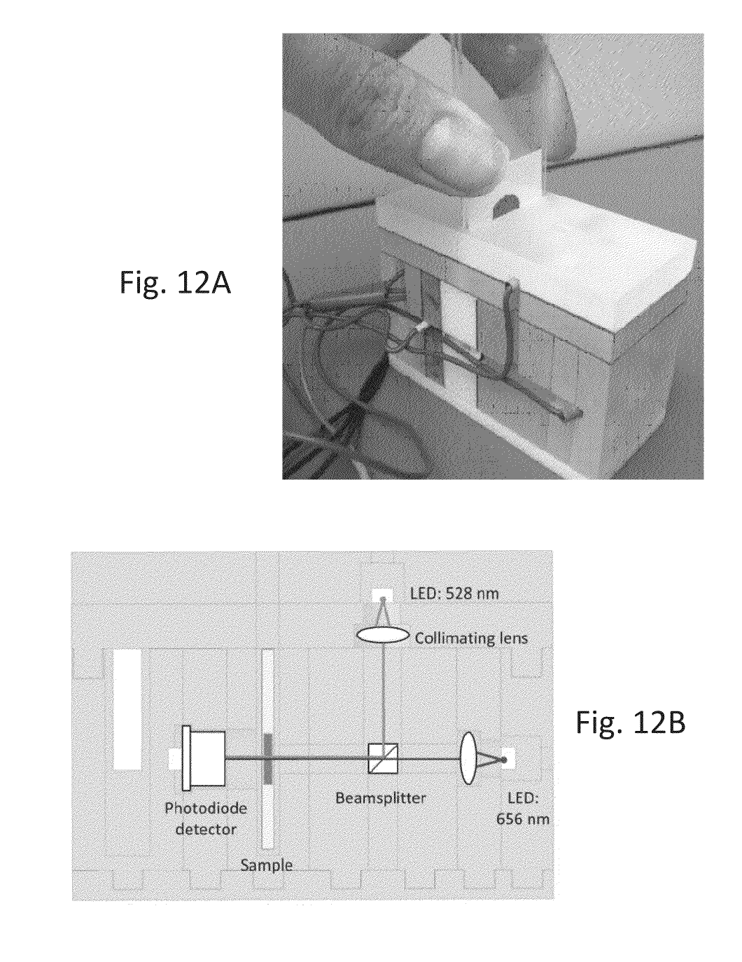

Example implementations of the present disclosure feature a reader to replace the spectrometer. Example of this reader are shown in FIGS. 12A and 12B. FIG. 12B shows a functional schematic diagram of the example reader. The example reader of FIG. 12B includes a first LED with a wavelength of about 656 nm and a second LED with a wavelength of about 528 nm. The outputs of the first and second LEDs pass though a first collimating lens and a second collimating lens before going to a beamsplitter. The combined signal is send through a sample and finally onto a photodiode detector.

FIG. 12A is a photograph of the completed reader. In this photograph, a sample is being placed into the reader for operation.

Light emitted from two LED diodes (HyperRed, λ=656±25 nm, 720-LHW5AM1T3T1LZ from Mouser Electronics (Mansfield, Tex.) and TrueGreen, 2, λ=528±33 nm, 720-LTWSSMJXKX36Z from Mouser Electronics) is collimated by plastic aspheric lenses (EFL=3.3 mm, NA=0.4, CAY033 from ThorLabs (Newton, N.J.)) and, after passing through 50/50 beamsplitter (BS007 from ThorLabs), is directed onto the sample. Light transmitted by the sample is detected by a broad-band photodiode (wavelength range: 350-1100 nm, FDS100 from ThorLabs) placed behind the sample chamber. In certain example implantations the electronic components of the system are connected to NI USB-6008 data acquisition module.

Samples may be inserted between two glass slides to protect the reader from biohazards; the glass slides may be reused. The chromatography paper may be cut such that aligning it with the bottom edge of the slide ensures proper alignment of the blood sample with the optics of the low-cost reader.

A LabVIEW program may take a one second baseline reading with both LEDs off to enable subtraction of ambient light, a two second reading with the hemoglobin absorption LED on, and a two second reading with the control LED on.

In certain example implementations, data is collected from the same samples in the training and validation sets described herein at 90 second after spotting. Data from the training set may be used to develop an algorithm relating the difference in transmission at 528 nm and 656 nm to the hemoglobin concentration as determined by the HemoCue. The algorithm was then used to predict the hemoglobin concentration for the 20 samples in the validation set using the concentration determined with the HemoCue as a comparison standard.

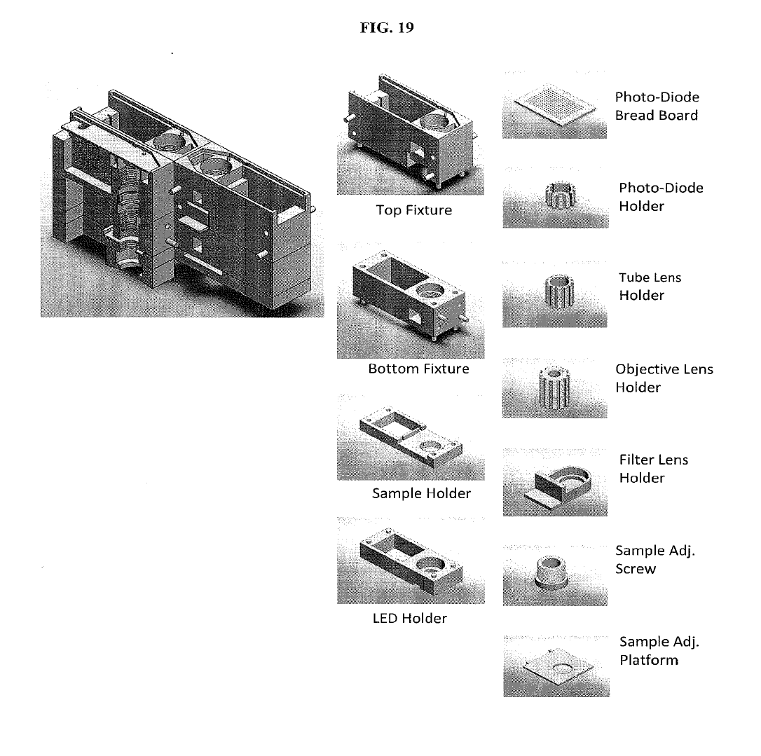

The apparatus of the present disclosure may a plurality of individual parts, as shown in FIG. 19. One or more of the parts may be fabricated using injection molding or other fabrication techniques. Certain example implementations include one or more top fixtures. Certain example implementations include one or more bottom fixtures. Certain example implementations include one or more sample holders. Example sample holders can be inserted and removed from a completed assembly so that a new sample may be introduced into the reader during operation. Certain example implementations include one or more photo diode boards. Certain example implementations include one or more photo diode holders. Certain example implementations include one or more tube lens holders. Certain example implementations include one or more objective lens holders. Certain example implementations include one or more filter lens holders. Certain example implementations include one or more LED Holders. Certain example implementations include one or more sample adjustment screws. Certain example implementations include one or more sample adjustment platforms.

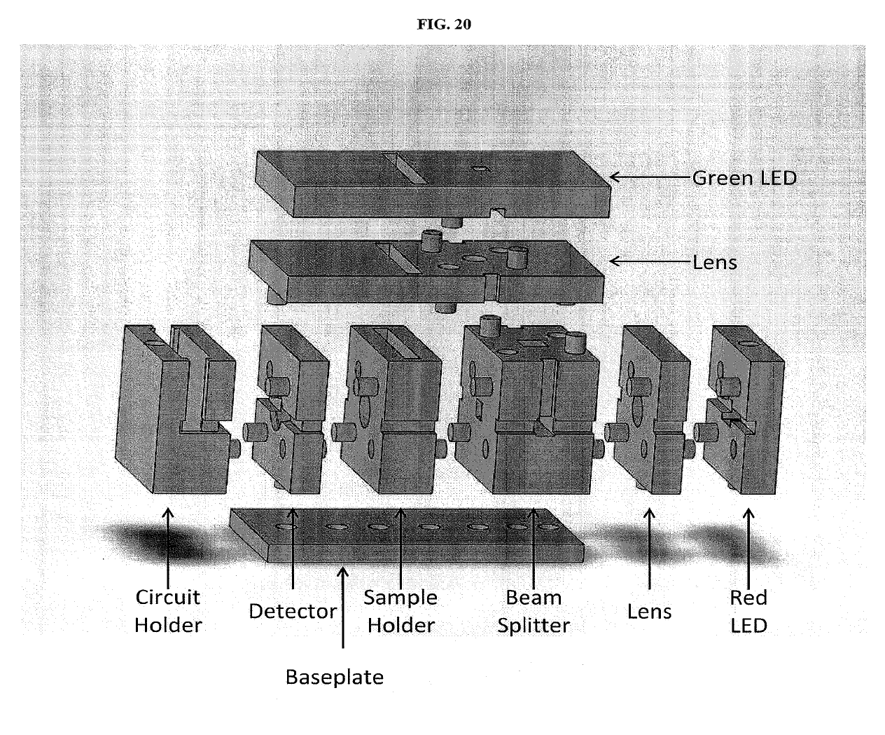

These individually manufactured parts may fit together as shown in Figure. 20. In that figure, a baseplate module is provided to hold other components. A circuit holder module is provided that is configured to be mated with the baseplate module and a detector modules. The detector module is configured to be mated with the baseplate and a sample holder module. The sample holder module is configured to be mated with the baseplate module, the detector module, and a beam splitter module. The beam splitter module is configured to be mated with the baseplate module, the sample holder module, and a lens module. The lens module is configured to be mated with the baseplate module, the beam splitter module, and an LED module. In certain implementations, the LED module is to hold a red LED, in other implementations, the LED module is to hold a blue LED. In still other implementations, the LED module is to hold a greed LED. As shown in FIG. 20, a second lens module is provided to be coupled to the assembled circuit holder module, detector module, sample holder module, beam splitter module, lens module, and LED module. A second LED module is configured to be coupled with the second lens module. In certain implementations, the second LED module is to hold a red LED, in other implementations, the second LED module is to hold a blue LED. In still other implementations, the second LED module is to hold a greed LED.

In certain implementations, the modules are modular to allow one or more components to be swapped out and the optical system to be customized. The low-cost reader my also be made to prevent disassembly, such as in cases where the reader will only be used for one type of test (e.g. hemoglobin concentration).

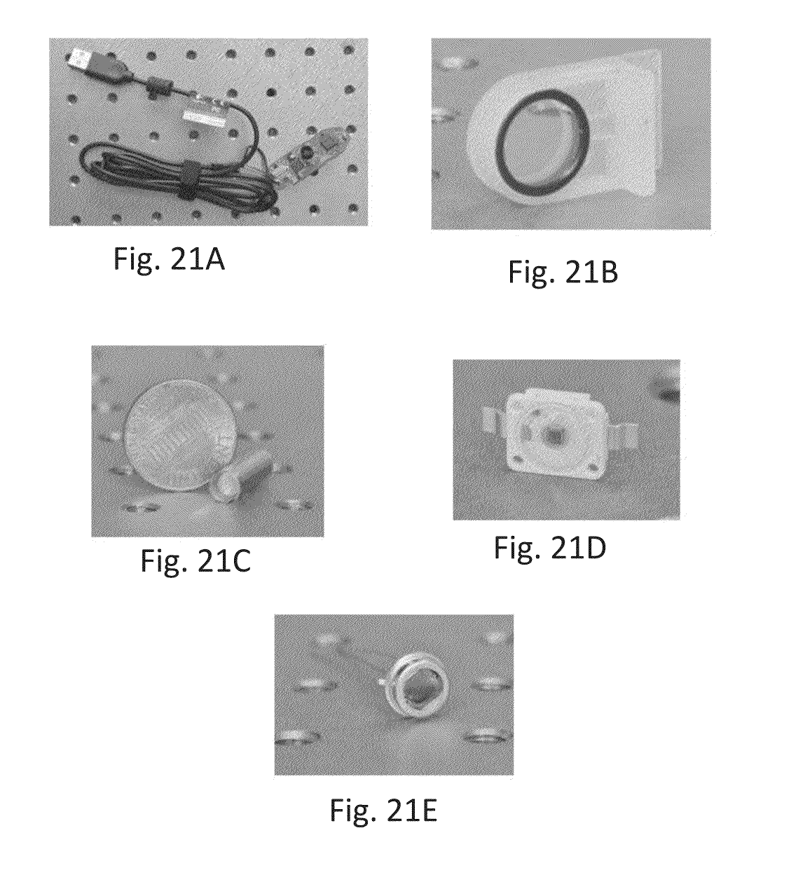

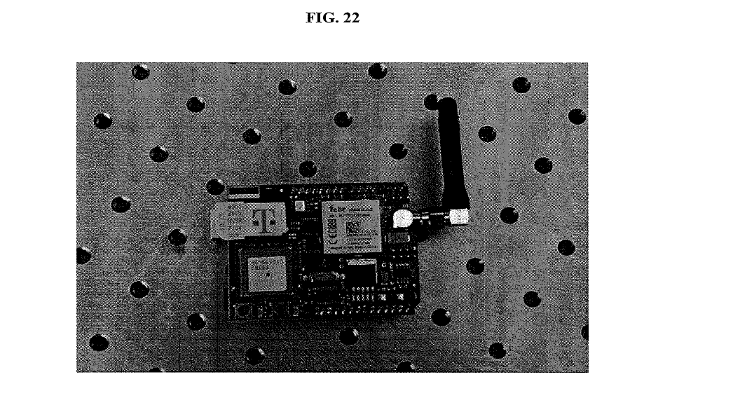

Certain implementations feature one more optical elements such as those shown in FIGS. 21B and 21C. Example optical element may feature one or more standardized optical parameters, based on the application. For example, one more optical elements may have a numerical aperture (NA) of 0.1, 0.25, or 0.5. This may allow for a broad range of imaging and detection applications. For example, one more optical elements may have a magnifications of 2, 4, 8, or other magnifications. Certain optical elements are system tolerance to minimize the amount of alignment necessary.

Certain implementations feature one or more electrical detector and one more electrical sources, which as shown in FIGS. 21A, 21D, 21E. As shown in FIG. 21A, certain components may include a USB readout capability so that results may be read by and stored on a computer with USB capability.

Certain implementations include a communication module, such as the module shown in FIG. 22. Figure shows an example wireless communication device that may be used to communicate and record results made by the reader. Example communications modules may use broadly accessible standards. For example, the module may be configured to use GSM or 802.11 wireless networking protocols. Example implementations may feature multiple communication or recording options. For example, certain implementations feature one or more of voice, text messaging (for example, by SMS), GPS, GPRS, TCP/IP, Fax, and pictures (for example, transmission by MMS).

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A readout and signal transduction (ROST) component, comprising:
a universal fixture adapted to receive and align one or more optical elements and one or more opto-mechanical elements, wherein the optical elements and one or more opto-mechanical elements are stacked in the universal fixture;
one or more optical elements configured for integration into the universal fixture; and
one or more opto-mechanical elements configured for integration into the universal fixture.

2. The readout and signal transduction (ROST) component of claim 1, wherein the universal fixture comprises one or more cylindrical tube and wherein the one or more optical elements and one or more opto-mechanical elements are configured to be stacked in cylindrical tubes.

3. The readout and signal transduction (ROST) component of claim 1, further comprising:
a sample platform to accept and hold a sample for analysis.

4. The readout and signal transduction (ROST) component of claim 1, further comprising:
an illumination unit comprising one or more of an LED and a laser, wherein the illumination unit is coupled to the universal fixture.

5. The readout and signal transduction (ROST) component of claim 1, further comprising:
a point detector, wherein the point detector is coupled to the universal fixture

6. The readout and signal transduction (ROST) component of claim 1, further comprising:
array detector coupled to the universal fixture.

7. The readout and signal transduction (ROST) component of claim 1, wherein the one or more optical elements comprise one or more elements selected from the group consisting of:
a condenser lens;
a collective lens;
a relay lens assembly of one or more lenses;
a re-imaging assembly of lenses;
a relay component;
a prism;
a grating;
a filter plate;
a polarizer;
a compensator;
a beam splitter; and
a pinhole grid.

8. The readout and signal transduction (ROST) component of claim 1, wherein the one or more opto-mechanical elements comprise one or more elements selected from the group consisting of:
a self-centering spring;
a kinematic component;
a spacer;
a mounting barrel.

9. The readout and signal transduction (ROST) component of claim 1, wherein the universal fixture, optical elements, and opto-mechanical elements are configured to perform sample interrogation based on transmission mode.

10. The readout and signal transduction (ROST) component of claim 1, wherein the universal fixture, optical elements, and opto-mechanical elements are configured to perform sample interrogation based on light scattering mode.

11. The readout and signal transduction (ROST) component of claim 1, wherein the universal fixture, optical elements, and opto-mechanical elements are configured to perform sample interrogation based on fluorescence mode.

12. The readout and signal transduction (ROST) component of claim 1, wherein the universal fixture, optical elements, and opto-mechanical elements are configured to perform sample interrogation based on spectrophotometry.

13. The readout and signal transduction (ROST) component of claim 1, wherein the universal fixture, optical elements, and opto-mechanical elements are configured to perform simultaneous analysis of multiple analytes with different fluorescence emissions.

14. A method of analyzing a sample, comprising:
providing a readout and signal transduction (ROST) component, comprising:
a universal fixture adapted to receive and align one or more optical elements and one or more opto-mechanical elements, wherein the optical elements and one or more opto-mechanical elements are stacked in the universal fixture;
one or more optical elements configured for integration into the universal fixture;
one or more opto-mechanical elements configured for integration into the universal fixture; and
a sample platform to accept and hold a sample for analysis; and
putting an sample on the sample platform;
analyzing the sample using the ROST component.
